(19) United States
(12) Patent Application Publication
(10) Pub. No.: US 2003/0224970 A1
(43) Pub. Date: Dec. 4, 2003

(54) Methods for the identification of inhibitors of S-adenosylmethionine decarboxylase as antibiotics

(76) Inventors: Sanjoy Mahanty, Chapel Hill, NC (US); Ryan Heiniger, Raleigh, NC (US); Amy Skalchunes, Raleigh, NC (US); Huaqin Pan, Apex, NC (US); Rex Tarpey, Apex, NC (US); Jeffrey Shuster, Chapel Hill, NC (US); Matthew M. Tanzer, Durham, NC (US); Lisbeth Hamer, Durham, NC (US); Kiichi Adachi, Tokyo (JP); Todd M. DeZwaan, Apex, NC (US); Sze-Chung Lo, Shun Lee Estate (HK); Maria Victoria Montenegro-Chamorro, Morrisville, NC (US); Sheryl Frank, Durham, NC (US); Blaise Darveaux, Hillsborough, NC (US)

Correspondence Address: PARADIGM GENETICS, INC, 108 ALEXANDER DRIVE, P O BOX 14528, RTP, NC, 27709-4528, US

(21) Appl. No.: 10/436,327

(22) Filed: May 12, 2003

Related U.S. Application Data
(60) Provisional application No. 60/381,223, filed on May 17, 2002.

(51) Int. Cl. A61K 31/00; C12Q 1/48; C07H 21/04; C12Q 1/18; C12N 9/10

(52) U.S. Cl. 514/1; 435/15; 435/32; 435/193; 536/23.2

(57) ABSTRACT

The present inventors have discovered that S-adenosylmethionine decarboxylase (“SPE2”) is essential for normal fungal pathogenicity. Specifically, the inhibition of S-adenosylmethionine decarboxylase gene expression in fungi results in greatly reduced pathogenicity. Thus, S-adenosylmethionine decarboxylase is useful as a target for the identification of antibiotics, preferably antifungals. Accordingly, the present invention provides methods for the identification of compounds that inhibit S-adenosylmethionine decarboxylase expression or activity. The methods of the invention are useful for the identification of antibiotics, preferably antifungals.

### RELATED APPLICATIONS

This application claims the benefit of U.S. Application No. 60/381,223 filed May 17, 2002, herein incorporated in its entirety by reference.

### FIELD OF THE INVENTION

The invention relates generally to methods for the identification of antibiotics, preferably antifungals that affect the biosynthesis of polyamine.

### BACKGROUND OF THE INVENTION

Filamentous fungi are the causal agents responsible for many serious pathogenic infections of plants and animals. Since fungi are eukaryotes, and thus more similar to their host organisms than, for example bacteria, the treatment of infections by fungi poses special risks and challenges not encountered with other types of infections. One such fungus is Magnaporthe grisea, the fungus that causes rice blast disease. It is an organism that poses a significant threat to food supplies worldwide. Other examples of plant pathogens of economic importance include the pathogens in the genera Agaricus, Alternaria, Anisogramma, Anthracoidea, Antrodia, Apiognomonia, Apiosporina, Armillaria, Ascochyta, Aspergillus, Bipolaris, Bjerkandera, Botryosphaeria, Botrytis, Ceratobasidium, Ceratocystis, Cercospora, Cercosporidium, Cerotelium, Cerrena, Chondrostereum, Chryphonectria, Chrysomyxa, Cladosporium, Claviceps, Cochliobolus, Coleosporium, Colletotrichium, Colletotrichum, Corticium, Corynespora, Cronartium, Cryphonectria, Cryptosphaeria, Cyathus, Cymadothea, Cytospora, Daedaleopsis, Diaporthe, Didymella, Diplocarpon, Diplodia, Discohainesia, Discula, Dothistroma, Drechslera, Echinodontium, Elsinoe, Endocronartium, Endothia, Entyloma, Epichloe, Erysiphe, Exobasidium, Exserohilum, Fomes, Fomitopsis, Fusarium, Gaeumannomyces, Ganoderma, Gibberella, Gloeocercospora, Gloeophyllum, Gloeoporus, Glomerella, Gnomoniella, Guignardia, Gymnosporangium, Helminthosporium, Herpotrichia, Heterobasidion, Hirschioporus, Hypodermella, Inonotus, Irpex, Kabatiella, Kabatina, Laetiporus, Laetisaria, Lasiodiplodia, Laxitextum, Leptographium, Leptosphaeria, Leptosphaerulina, Leucytospora, Linospora, Lophodermella, Lophodermium, Macrophomina, Magnaporthe, Marssonina, Melampsora, Melampsorella, Meria, Microdochium, Microsphaera, Monilinia, Monochaetia, Morchella, Mycosphaerella, Myrothecium, Nectria, Nigrospora, Ophiosphaerella, Ophiostoma, Penicillium, Perenniporia, Peridermium, Pestalotia, Phaeocryptopus, Phaeolus, Phakopsora, Phellinus, Phialophora, Phoma, Phomopsis, Phragmidium, Phyllachora, Phyllactinia, Phyllosticta, Phymatotrichopsis, Pleospora, Podosphaera, Pseudopeziza, Pseudoseptoria, Puccinia, Pucciniastrum, Pyricularia, Rhabdocline, Rhizoctonia, Rhizopus, Rhizosphaera, Rhynchosporium, Rhytisma, Schizophyllum, Schizopora, Scirrhia, Sclerotinia, Sclerotium, Scytinostroma, Septoria, Setosphaera, Sirococcus, Spaerotheca, Sphaeropsis, Sphaerotheca, Sporisorium, Stagonospora, Stemphylium, Stenocarpella, Stereum, Taphrina, Thielaviopsis, Tilletia, Trametes, Tranzschelia, Trichoderma, Tubakia, Typhula, Uncinula, Urocystis, Uromyces, Ustilago, Valsa, Venturia, Verticillium, Xylaria, and others. Related organisms are classified in the oomycetes classification and include the genera Albugo, Aphanomyces, Bremia, Peronospora, Phytophthora, Plasmodiophora, Plasmopara, Pseudoperonospora, Pythium, Sclerophthora, and others. Oomycetes are significant plant pathogens and are sometimes classified along with the true fungi.

Human diseases caused by filamentous fungi include life-threatening lung and disseminated diseases, often resulting from infections by Aspergillus fumigatus. Other fungal diseases in animals are caused by fungi in the genera, Fusarium, Blastomyces, Microsporum, Trichophyton, Epidermophyton, Candida, Histoplamsa, Pneumocystis, Cryptococcus, other Aspergilli, and others. The control of fungal diseases in plants and animals is usually mediated by chemicals that inhibit the growth, proliferation, and/or pathogenicity of the fungal organisms. To date, there are less than twenty known modes-of-action for plant protection fungicides and human antifungal compounds. A pathogenic organism has been defined as an organism that causes, or is capable of causing disease. Pathogenic organisms propagate on or in tissues and may obtain nutrients and other essential materials from their hosts. A substantial amount of work concerning filamentous fungal pathogens has been performed with the human pathogen, Aspergillus fumigatus. Shibuya et al. (Shibuya, K., M. Takaoka, et al. (1999) Microb Pathog 27: 123-31 (PMID: 10455003)) have shown that the deletion of either of two suspected pathogenicity related genes encoding an alkaline protease or a hydrophobin (rodlet) respectively, did not reduce mortality of mice infected with these mutant strains. Smith et al. (Smith, J. M., C. M. Tang, et al. (1994) Infect Immun 62: 5247-54 (PMID: 7960101)) showed similar results with alkaline protease and the ribotoxin restrictocin; Aspergillus fumigatus strains mutated for either of these genes were fully pathogenic to mice. Reichard et al. (Reichard, U., M. Monod, et al. (1997) J Med Vet Mycol 35: 189-96(PMID: 9229335)) showed that deletion of the suspected pathogenicity gene encoding aspergillopepsin (PEP) in Aspergillus fumigatus had no effect on mortality in a guinea pig model system, and Aufauvre-Brown et al (Aufauvre-Brown, A., E. Mellado, et al. (1997) Fungal Genet Biol 21: 141-52 (PMID: 9073488)) showed no effects of a chitin synthase mutation on pathogenicity. However, not all experiments produced negative results. Ergosterol is an important membrane component found in fungal organisms. Pathogenic fungi that lack key enzymes in this biochemical pathway might be expected to be non-pathogenic since neither the plant nor animal hosts contain this particular sterol. Many antifungal compounds that affect this biochemical pathway have been previously described. (U.S. Pat. Nos. 4,920,109; 4,920,111; 4,920,112; 4,920,113; and 4,921,844; Fungicides in Crop Protection Cambridge, University Press (1990)). D'Enfert et al. (D'Enfert, C., M. Diaquin, et al. (1996) Infect Immun 64: 4401-5 (PMID: 8926121)) showed that an Aspergillus fumigatus strain mutated in an orotidine 5′-phosphate decarboxylase gene was entirely non-pathogenic in mice, and Brown et al. (Brown, J. S., A. Aufauvre-Brown, et al. (2000) Mol Microbiol 36: 1371-80 (PMID: 10931287)) observed a non-pathogenic result when genes involved in the synthesis of para-aminobenzoic acid were mutated. Some specific target genes have been described as having utility for the screening of inhibitors of plant pathogenic fungi. U.S. Pat. No. 6,074,830, issued to Bacot et al. describes the use of 3,4-dihydroxy-2-butanone 4-phosphate synthase, and U.S. Pat. No. 5,976,848, issued to Davis et al. describes the use of dihydroorotate dehydrogenase for potential screening purposes.

There are also a number of papers that report less clear results, showing neither full pathogenicity nor non-pathogenicity of mutants. Hensel et al. (Hensel, M., H. N. Arst, Jr., et al. (1998) Mol Gen Genet 258: 553-7 (PMID: 9669338)) showed only moderate effects of the deletion of the area transcriptional activator on the pathogenicity of Aspergillus fumigatus.

Therefore, it is not currently possible to determine which specific growth materials may be readily obtained by a pathogen from its host, and which materials may not. Magnaporthe grisea that cannot synthesize their own polyamine are observed to exhibit reduced pathogenicity on their host organism, producing smaller lesions that fail to spread across a leaf's surface. To date nothing contained within the literature identifies an anti-pathogenic effect of the knock-out, over-expression, antisense expression, or inhibition of the genes or gene products involved in polyamine biosynthesis in Magnaporthe. Thus, it has not been shown that the de novo biosynthesis of polyamine is essential for fungal Magnaporthe pathogenicity, however, is has been shown to be essential for pathogenicity of other fungal species. An application related to the present application entitled, “Methods for the Identification of Inhibitors of Putrescine aminopropyltransferase as Antibiotics,” U.S. Application Serial No. 60/381,151, incorporated herein by reference, shows that the disruption of polyamine biosynthesis as the result of a disruption of the gene encoding the enzyme activity, Putrescine aminopropyltransferase, results in a non-pathogenic phenotype for M. grisea. Thus, it would be desirable to determine the utility of the enzymes involved in polyamine biosynthesis for evaluating antibiotic compounds, especially fungicides. If a fungal biochemical pathway or specific gene product in that pathway is shown to be required for fungal pathogenicity, various formats of in vitro and in vivo screening assays may be put in place to discover classes of chemical compounds that react with the validated target gene, gene product, or biochemical pathway, and are thus candidates for antifungal, biocide, and biostatic materials.

### SUMMARY OF THE INVENTION

The present inventors have discovered that in vivo disruption of the gene encoding S-adenosylmethionine decarboxylase in Magnaporthe grisea prevents or inhibits the pathogenicity of the fungus. Thus, the present inventors have discovered that S-adenosylmethionine decarboxylase is essential for normal rice blast pathogenicity, and can be used as a target for the identification of antibiotics, preferably fungicides. Accordingly, the present invention provides methods for the identification of compounds that inhibit S-adenosylmethionine decarboxylase expression or activity. The methods of the invention are useful for the identification of antibiotics, preferably fungicides.

### BRIEF DESCRIPTION OF THE FIGURES

FIG. 1 shows the reaction performed by S-adenosylmethionine decarboxylase (SPE2). The Substrate/Product is S-Adenosyl-L-methionine and the Products/Substrates are (5-Deoxy-5-adenosyl) (3-aminopropyl) methylsulfonium salt and CO₂. The function of the S-adenosylmethionine decarboxylase enzyme is the interconversion of S-Adenosyl-L-methionine to (5-Deoxy-5-adenosyl) (3-aminopropyl) methylsulfonium salt and CO₂. This reaction is part of the polyamine biosynthesis pathway.

FIG. 2 shows a digital image showing the effect of SPE2 gene disruption on Magnaporthe grisea pathogenicity using whole plant infection assays. Rice variety CO39 was inoculated with wild-type and the transposon insertion strains, KO1-1 and KO1-36. Leaf segments were imaged at five days post-inoculation.

### DETAILED DESCRIPTION OF THE INVENTION

Unless otherwise indicated, the following terms are intended to have the following meanings in interpreting the present invention.

The term “antibiotic” refers to any substance or compound that when contacted with a living cell, organism, virus, or other entity capable of replication, results in a reduction of growth, viability, or pathogenicity of that entity.

The term “antipathogenic,” as used herein, refers to a mutant form of a gene that inactivates a pathogenic activity of an organism on its host organism or substantially reduces the level of pathogenic activity, wherein “substantially” means a reduction at least as great as the standard deviation for a measurement, preferably a reduction to 50% activity, more preferably a reduction of at least one magnitude, i.e. to 10% activity. The pathogenic activity affected may be an aspect of pathogenic activity governed by the normal form of the gene, or the pathway the normal form of the gene functions on, or the pathogenic activity of the organism in general. “Antipathogenic” may also refer to a cell, cells, tissue, or organism that contains the mutant form of a gene; a phenotype associated with the mutant form of a gene, and/or associated with a cell, cells, tissue, or organism that contain the mutant form of a gene.

The term “bDNA” refers to branched DNA.

The term “binding” refers to a non-covalent or a covalent interaction, preferably non-covalent, that holds two molecules together. For example, two such molecules could be an enzyme and an inhibitor of that enzyme. Non-covalent interactions include hydrogen bonding, ionic interactions among charged groups, van der Waals interactions and hydrophobic interactions among nonpolar groups. One or more of these interactions can mediate the binding of two molecules to each other.

The term “biochemical pathway” or “pathway” refers to a connected series of biochemical reactions normally occurring in a cell, or more broadly a cellular event such as cellular division or DNA replication. Typically, the steps in such a biochemical pathway act in a coordinated fashion to produce a specific product or products or to produce some other particular biochemical action. The pathway therefore requires the expression product of a gene in cases where the absence of that expression product either directly or indirectly prevents the completion of one or more steps in that pathway, thereby preventing or significantly reducing the production of one or more normal products or effects of that pathway. Thus, an agent specifically inhibits such a biochemical pathway requiring the expression product of a particular gene if the presence of the agent stops or substantially reduces the completion of the series of steps in that pathway. Such an agent may, but does not necessarily, act directly on the expression product of that particular gene.

As used herein, the term “conditional lethal” refers to a mutation permitting growth and/or survival only under special growth or environmental conditions.

As used herein, the term “cosmid” refers to a hybrid vector used in gene cloning that includes a cos site (from the lambda bacteriophage). In some cases, the cosmids of the invention comprise drug resistance marker genes and other plasmid genes. Cosmids are especially suitable for cloning large genes or multigene fragments.

“Fungi” (singular: fungus) refers to whole fungi, fungal organs and tissues (e.g., asci, hyphae, pseudohyphae, rhizoid, sclerotia, sterigmata, spores, sporodochia, sporangia, synnemata, conidia, ascostroma, cleistothecia, mycelia. perithecia, basidia and the like), spores, fungal cells and the progeny thereof. Fungi are a group of organisms (about 50,000 known species), including, but not limited to, mushrooms, mildews, moulds, yeasts, etc., comprising the kingdom Fungi. Fungi exist as single cells or a multicellular body called a mycelium, which consists of filaments known as hyphae. Most fungal cells are multinucleate and have cell walls composed chiefly of chitin. Fungi exist primarily in damp situations on land, and lacking the ability to manufacture their own food by photosynthesis due to the absence of chlorophyll, are either parasites on other organisms or saprotrophs feeding on dead organic matter. Principal criteria used in classification are the nature of the spores produced and the presence or absence of cross walls within the hyphae. Fungi are distributed worldwide in terrestrial, freshwater, and marine habitats. Some fungi live in the soil. Many pathogenic fungi cause disease in animals and man or in plants, while some saprotrophs are destructive to timber, textiles, and other materials. Some fungi form associations with other organisms, most notably with algae to form lichens.

As used herein, the term “fungicide,” “antifungal,” or “antimycotic” refers to an antibiotic substance or compound that kills or suppresses the growth, viability, or pathogenicity of at least one fungus, fungal cell, fungal tissue or spore.

In the context of this disclosure, “gene” should be understood to refer to a unit of heredity. Each gene is composed of a linear chain of deoxyribonucleotides that can be referred to by the sequence of nucleotides forming the chain. Thus, “sequence” is used to indicate both the ordered listing of the nucleotides which form the chain, and the chain having that sequence of nucleotides. “Sequence” is used in the similar way in referring to RNA chains, linear chains made of ribonucleotides. The gene may include regulatory and control sequences, sequences which can be transcribed into an RNA molecule, and may contain sequences with unknown function. The majority of the RNA transcription products are messenger RNAs (mRNAs), which include sequences which are translated into polypeptides and may include sequences which are not translated. It should be recognized that small differences in nucleotide sequence for the same gene can exist between different fungal strains, or even within a particular fungal strain, without altering the identity of the gene.

As used in this disclosure, the terms “growth” or “cell growth” of an organism refer to an increase in mass, density, or number of cells of the organism. Common methods for the measurement of growth include the determination of the optical density of a cell suspension, the counting of the number of cells in a fixed volume, the counting of the number of cells by measurement of cell division, the measurement of cellular mass or cellular volume, and the like.As used in this disclosure, the term “growth conditional phenotype” indicates that a fungal strain having such a phenotype exhibits a significantly greater difference in growth rates in response to a change in one or more of the culture parameters than an otherwise similar strain not having a growth conditional phenotype. Typically, a growth conditional phenotype is described with respect to a single growth culture parameter, such as temperature. Thus, a temperature (or heat-sensitive) mutant (i.e., a fungal strain having a heat-sensitive phenotype) exhibits significantly different growth, and preferably no growth, under non-permissive temperature conditions as compared to growth under permissive conditions. In addition, such mutants preferably also show intermediate growth rates at intermediate, or semi-permissive, temperatures. Similar responses also result from the appropriate growth changes for other types of growth conditional phenotypes.

As used in this disclosure, the term “growth conditional phenotype” indicates that a fungal strain having such a phenotype exhibits a significantly greater difference in growth rates in response to a change in one or more of the culture parameters than an otherwise similar strain not having a growth conditional phenotype. Typically, a growth conditional phenotype is described with respect to a single growth culture parameter, such as temperature. Thus, a temperature (or heat-sensitive) mutant (i.e., a fungal strain having a heat-sensitive phenotype) exhibits significantly different growth, and preferably no growth, under non-permissive temperature conditions as compared to growth under permissive conditions. In addition, such mutants preferably also show intermediate growth rates at intermediate, or semi-permissive, temperatures. Similar responses also result from the appropriate growth changes for other types of growth conditional phenotypes.

As used herein, the term “heterologous SPE2” means either a nucleic acid encoding a polypeptide or a polypeptide, wherein the polypeptide has at least 50%, 55%, 60%, 65%, 70%, 75%, 80%, 85%, 90%, 91%, 92%, 93%, 94%, 95%, 96%, 99% sequence identity or each integer unit of sequence identity from 50-100% in ascending order to M. grisea SPE2 protein (SEQ ID NO:3) and at least 10%, 25%, 50%, 75%, 80%, 90%, 95%, or 99% activity or each integer unit of activity from 10-100% in ascending order of the activity of M. grisea SPE2 protein (SEQ ID NO:3). One example of a heterologous SPE2 is S-adenosylmethionine decarboxylase from Neurospora crassa (GenBank Accession number 4929540).

As used herein, the term “His-Tag” refers to an encoded polypeptide consisting of multiple consecutive histidine amino acids.

As used herein, the terms “hph,” “hygromycin B phosphotransferase,” and “hygromycin resistance gene” refer to a hygromycin phosphotransferase gene or gene product.

As used herein, the term “imperfect state” refers to a classification of a fungal organism having no demonstrable sexual life stage.

The term “inhibitor,” as used herein, refers to a chemical substance that inactivates the enzymatic activity of S-adenosylmethionine decarboxylase or substantially reduces the level of enzymatic activity, wherein “substantially” means a reduction at least as great as the standard deviation for a measurement, preferably a reduction to 50% activity, more preferably a reduction of at least one magnitude, i.e. to 10% activity. The inhibitor may function by interacting directly with the enzyme, a cofactor of the enzyme, the substrate of the enzyme, or any combination thereof.

A polynucleotide may be “introduced” into a fungal cell by any means known to those of skill in the art, including transfection, transformation or transduction, transposable element, electroporation, particle bombardment, infection, and the like. The introduced polynucleotide may be maintained in the cell stably if it is incorporated into a non-chromosomal autonomous replicon or integrated into the fungal chromosome. Alternatively, the introduced polynucleotide may be present on an extra-chromosomal non-replicating vector and be transiently expressed or transiently active.

As used herein, the term “knockout” or “gene disruption” refers to the creation of organisms carrying a null mutation (a mutation in which there is no active gene product), a partial null mutation or mutations, or an alteration or alterations in gene regulation by interrupting a DNA sequence through insertion of a foreign piece of DNA. Usually the foreign DNA encodes a selectable marker.

As used herein, the term “mutant form” of a gene refers to a gene which has been altered, either naturally or artificially, by changing the base sequence of the gene. The change in the base sequence may be of several different types, including changes of one or more bases for different bases, deletions, and/or insertions, such as by a transposon. In contrast, a normal form of a gene (wild-type) is a form commonly found in natural populations of an organism. Commonly a single form of a gene will predominate in natural populations. In general, such a gene is suitable as a normal form of a gene, however, other forms which provide similar functional characteristics may also be used as a normal gene. In particular, a normal form of a gene does not confer a growth conditional phenotype on the strain having that gene, while a mutant form of a gene suitable for use in these methods does provide such a growth conditional phenotype.

As used herein, the term “Ni-NTA” refers to nickel sepharose.

As used herein, a “normal” form of a gene (wild-type) is a form commonly found in natural populations of an organism. Commonly a single form of a gene will predominate in natural populations. In general, such a gene is suitable as a normal form of a gene, however, other forms which provide similar functional characteristics may also be used as a normal gene. In particular, a normal form of a gene does not confer a growth conditional phenotype on the strain having that gene, while a mutant form of a gene suitable for use in these methods does provide such a growth conditional phenotype.

As used herein, the term “pathogenicity” refers to a capability of causing disease and/or degree of capacity to cause disease. The term is applied to parasitic micro-organisms in relation to their hosts. As used herein, “pathogenicity,” “pathogenic,” and the like, encompass the general capability of causing disease as well as various mechanisms and structural and/or functional deviations from normal used in the art to describe the causative factors and/or mechanisms, presence, pathology, and/or progress of disease, such as virulence, host recognition, cell wall degradation, toxin production, infection hyphae, penetration peg production, appressorium production, lesion formation, sporulation, and the like.

The “percent (%) sequence identity” between two polynucleotide or two polypeptide sequences is determined according to either the BLAST program (Basic Local Alignment Search Tool, (Altschul, S. F. et al., 215 J. Mol. Biol. 403 (1990) (PMID: 2231712)) or using Smith Waterman Alignment (T. F. Smith & M. S. Waterman 147 J. Mol. Biol. 195 (1981) (PMID: 7265238)). It is understood that for the purposes of determining sequence identity when comparing a DNA sequence to an RNA sequence, a thymine nucleotide is equivalent to a uracil nucleotide.

By “polypeptide” is meant a chain of at least two amino acids joined by peptide bonds. The chain may be linear, branched, circular or combinations thereof. The polypeptides may contain amino acid analogs and other modifications, including, but not limited to glycosylated or phosphorylated residues.

As used herein, the term “proliferation” is synonymous to the term “growth.”

As used herein, the terms “S-adenosylmethionine decarboxylase (SPE2)” and “S-adenosylmethionine decarboxylase (SPE2) polypeptide” refer to an enzyme that catalyzes the reversible interconversion of S-adenosyl-L-methionine with (5-deoxy-5-adenosyl)(3-aminopropyl) methylsulfonium salt and CO₂. Although the protein and/or the name of the gene that encodes the protein may differ between species, the terms “SPE2” and “SPE2 gene product” are intended to encompass any polypeptide that catalyzes the reversible interconversion of S-adenosyl-L-methionine with (5-deoxy-5-adenosyl)(3-aminopropyl) methylsulfonium salt and CO₂.

As used herein, “semi-permissive conditions” are conditions in which the relevant culture parameter for a particular growth conditional phenotype is intermediate between permissive conditions and non-permissive conditions. Consequently, in semi-permissive conditions an organism having a growth conditional phenotype will exhibit growth rates intermediate between those shown in permissive conditions and non-permissive conditions. In general, such intermediate growth rate may be due to a mutant cellular component that is partially functional under semi-permissive conditions, essentially fully functional under permissive conditions, and is non-functional or has very low function under non-permissive conditions, where the level of function of that component is related to the growth rate of the organism. An intermediate growth rate may also be a result of a nutrient substance or substances that are present in amounts not sufficient for optimal growth rates to be achieved.

“Sensitivity phenotype” refers to a phenotype that exhibits either hypersensitivity or hyposensitivity.

The term “specific binding” refers to an interaction between S-adenosyl-methionine decarboxylase and a molecule or compound, wherein the interaction is dependent upon the primary amino acid sequence and/or the tertiary conformation of S-adenosylmethionine decarboxylase. An “SPE2 ligand” is an example of specific binding.

“Transform,” as used herein, refers to the introduction of a polynucleotide (single or double stranded DNA, RNA, or a combination thereof) into a living cell by any means. Transformation may be accomplished by a variety of methods, including, but not limited to, electroporation, polyethylene glycol mediated uptake, particle bombardment, agrotransformation, and the like. The transformation process may result in transient or stable expression of the transformed polynucleotide. By “stably transformed” is meant that the sequence of interest is integrated into a replicon in the cell, such as a chromosome or episome. Transformed cells encompass not only the end product of a transformation process, but also the progeny thereof which retain the polynucleotide of interest.

For the purposes of the invention, “transgenic” refers to any cell, spore, tissue or part, that contains all or part of at least one recombinant polynucleotide. In many cases, all or part of the recombinant polynucleotide is stably integrated into a chromosome or stable extra-chromosomal element, so that it is passed on to successive generations.

As used herein, the term “Tween 20” means sorbitan mono-9-octadecenoate poly(oxy-1,1-ethanediyl).

As used in this disclosure, the term “viability” of an organism refers to the ability of an organism to demonstrate growth under conditions appropriate for the organism, or to demonstrate an active cellular function. Some examples of active cellular functions include respiration as measured by gas evolution, secretion of proteins and/or other compounds, dye exclusion, mobility, dye oxidation, dye reduction, pigment production, changes in medium acidity, and the like.

The present inventors have discovered that disruption of the SPE2 gene and/or gene product inhibits the pathogenicity of Magnaporthe grisea. Thus, the inventors demonstrated that S-adenosylmethionine decarboxylase is a target for antibiotics, preferably antifungals.

The present invention provides methods for identifying compounds that inhibit SPE2 gene expression or biological activity of its gene product(s). Such methods include ligand binding assays, assays for enzyme activity, cell-based assays, and assays for SPE2 gene expression. Any compound that is a ligand for S-adenosylmethionine decarboxylase may have antibiotic activity. For the purposes of the invention, “ligand” refers to a molecule that will bind to a site on a polypeptide. The compounds identified by the methods of the invention are useful as antibiotics.

Thus, in one embodiment, the invention provides a method for identifying a test compound as a candidate for an antibiotic, comprising contacting an SPE2 polypeptide with a test compound and detecting the presence or absence of binding between the test compound and the SPE2 polypeptide, wherein binding indicates that the test compound is a candidate for an antibiotic.

SPE2 polypeptides of the invention have the amino acid sequence of a naturally occurring SPE2 found in a fungus, animal, plant, or microorganism, or have an amino acid sequence derived from a naturally occurring sequence. Preferably the SPE2 is a fungal SPE2. A cDNA encoding M. grisea SPE2 protein is set forth in SEQ ID NO:1, an M. grisea SPE2 genomic DNA is set forth in SEQ ID NO:2, and an M. grisea SPE2 polypeptide is set forth in SEQ ID NO:3. In one embodiment, the SPE2 is a Magnaporthe SPE2. Magnaporthe species include, but are not limited to, Magnaporthe rhizophila, Magnaporthe salvinii, Magnaporthe grisea and Magnaporthe poae and the imperfect states of Magnaporthe in the genus Pyricularia. Preferably, the Magnaporthe SPE2 is from Magnaporthe grisea.

In one embodiment, the invention provides a polypeptide consisting essentially of SEQ ID NO:3. For the purposes of the present invention, a polypeptide consisting essentially of SEQ ID NO:3 has at least 90% sequence identity with M. grisea SPE2 (SEQ ID NO:3) and at least 10% of the activity of SEQ ID NO:3. A polypeptide consisting essentially of SEQ ID NO:3 has at least 90%, 91%, 92%, 93%, 94%, 95%, 96%, 97%, 98%, or 99% sequence identity with SEQ ID NO:3 and at least 25%, 50%, 75%, or 90% of the activity of M grisea SPE2. Examples of polypeptides consisting essentially of SEQ ID NO:3 include, but are not limited to, polypeptides having the amino acid sequence of SEQ ID NO:3 with the exception that one or more of the amino acids are substituted with structurally similar amino acids providing a conservative amino acid substitution. Conservative amino acid substitutions are well known to those of skill in the art. Examples of polypeptides consisting essentially of SEQ ID NO:3 include polypeptides having 1, 2, or 3 conservative amino acid substitutions relative to SEQ ID NO:3. Other examples of polypeptides consisting essentially of SEQ ID NO:3 include polypeptides having the sequence of SEQ ID NO:3, but with truncations at either or both the 3′ and the 5′ end. For example, polypeptides consisting essentially of SEQ ID NO:3 include polypeptides having 1, 2, or 3 amino acids residues removed from either or both 3′ and 5′ ends relative to SEQ ID NO:3.

In various embodiments, the SPE2 can be from Powdery Scab (Spongospora subterranea), Grey Mould (Botrytis cinerea), White Rot (Armillaria mellea), Heartrot Fungus (Ganoderma adspersum), Brown-Rot (Piptoporus betulinus), Corn Smut (Ustilago maydis), Heartrot (Polyporus squamosus), Gray Leaf Spot (Cercospora zeae-maydis), Honey Fungus (Armillaria gallica), Root rot (Armillaria luteobubalina), Shoestring Rot (Armillaria ostoyae), Banana Anthracnose Fungus (Colletotrichum musae), Apple-rotting Fungus (Monilinia fructigena), Apple-rotting Fungus (Penicillium expansum), Clubroot Disease (Plasmodiophora brassicae), Potato Blight (Phytophthora infestans), Root pathogen (Heterobasidion annosum), Take-all Fungus (Gaeumannomyces graminis), Dutch Elm Disease (Ophiostoma ulmi), Bean Rust (Uromyces appendiculatus), Northern Leaf Spot (Cochliobolus carbonum), Milo Disease (Periconia circinata), Southern Corn Blight (Cochliobolus heterostrophus), Leaf Spot (Cochliobolus lunata), Brown Stripe (Cochliobolus stenospilus), Panama disease (Fusarium oxysporum), Wheat Head Scab Fungus (Fusarium graminearum), Cereal Foot Rot (Fusarium culmorum), Potato Black Scurf (Rhizoctonia solani), Wheat Black Stem Rust (Puccinia graminis), White mold (Sclerotinia sclerotiorum), and the like.

Fragments of a SPE2 polypeptide are useful in the methods of the invention. In one embodiment, the SPE2 fragments include an intact or nearly intact epitope that occurs on the biologically active wild-type SPE2. For example, the fragments comprise at least 10 consecutive amino acids of SPE2 set forth in SEQ ID NO:3. The fragments comprise at least 15, 20, 25, 30, 35, 40, 50, 60, 70, 80, 90, 100, 125, 150, 175, 200, 225, 250, 275, 300, 325, 350, 375, 400, 425, 450, or at least 475 consecutive amino acids residues of SPE2 set forth in SEQ ID NO:3. Fragments of heterologous SPE2s are also useful in the methods of the invention. For example, polypeptides having at least 50%, 60%, 70%, 80%, 90%, 95%, 96%, 97%, 98% or 99% sequence identity with at least 50 consecutive amino acid residues of SEQ ID NO:2 are useful in the methods of the invention. In one embodiment, the fragment is from a Magnaporthe SPE2. In an alternate embodiment, the fragment contains an amino acid sequence conserved among fungal SPE2s.

Polypeptides having at least 50% sequence identity with M. grisea SPE2 (SEQ ID NO:3) protein are also useful in the methods of the invention. In one embodiment, the sequence identity is at least 55%, 60%, 65%, 70%, 75%, 80%, 85%, 90%, 91%, 92%, 93%, 94%, 95%, 96%, 97%, 98% or 99%, or any integer from 50-100% sequence identity in ascending order with M. grisea SPE2 (SEQ ID NO:3) protein. In addition, it is preferred that polypeptides of the invention have at least 10% of the activity of M. grisea SPE2 (SEQ ID NO:3) protein. SPE2 polypeptides of the invention have at least 10%, 15%, 20%, 25%, 30%, 35%, 40%, 45%, 50%, 55%, 60%, 65%, 70%, 85% or at least 90% of the activity of M. grisea SPE2 (SEQ ID NO:3) protein.

Thus, in another embodiment, the invention provides a method for identifying a test compound as a candidate for a fungicide, comprising: contacting a test compound with at least one polypeptide selected from the group consisting of: a polypeptide consisting essentially of SEQ ID NO:3, a polypeptide having at least ten consecutive amino acids of an M. grisea SPE2 (SEQ ID NO:3) protein, a polypeptide having at least 50% sequence identity with an M. grisea SPE2 (SEQ ID NO:3) protein and at least 10% of the activity of an M. grisea SPE2 (SEQ ID NO:3) protein, and a polypeptide consisting of at least 50 amino acids having at least 50% sequence identity with an M. grisea SPE2 (SEQ ID NO:3) protein and at least 10% of the activity of an M. grisea SPE2 (SEQ ID NO:3) protein, and detecting the presence and/or absence of binding between the test compound and the polypeptide, wherein binding indicates that the test compound is a candidate for an antibiotic.

Any technique for detecting the binding of a ligand to its target may be used in the methods of the invention. For example, the ligand and target are combined in a buffer. Many methods for detecting the binding of a ligand to its target are known in the art, and include, but are not limited to, the detection of an immobilized ligand-target complex or the detection of a change in the properties of a target when it is bound to a ligand. For example, in one embodiment, an array of immobilized candidate ligands is provided. The immobilized ligands are contacted with a SPE2 protein or a fragment or variant thereof, the unbound protein is removed, and the bound SPE2 is detected. In a preferred embodiment, bound SPE2 is detected using a labeled binding partner, such as a labeled antibody. In an alternate preferred embodiment, SPE2 is labeled prior to contacting the immobilized candidate ligands. Preferred labels include fluorescent or radioactive moieties. Preferred detection methods include fluorescence correlation spectroscopy (FCS) and FCS-related confocal nanofluorimetric methods.

Once a compound is identified as a candidate for an antibiotic, it can be tested for the ability to inhibit SPE2 enzymatic activity. The compounds can be tested using either in vitro or cell based assays. Alternatively, a compound can be tested by applying it directly to a fungus or fungal cell, or expressing it therein, and monitoring the fungus or fungal cell for changes or decreases in growth, development, viability, pathogenicity, or alterations in gene expression. Thus, in one embodiment, the invention provides a method for determining whether a compound identified as an antibiotic candidate by an above method has antifungal activity, further comprising: contacting a fungus or fungal cells with said antifungal candidate and detecting a decrease in the growth, viability, or pathogenicity of said fungus or fungal cells.

By decrease in growth, is meant that the antifungal candidate causes at least a 10% decrease in the growth of the fungus or fungal cells, as compared to the growth of the fungus or fungal cells in the absence of the antifungal candidate. By a decrease in viability is meant that at least 20% of the fungal cells, or portion of the fungus contacted with the antifungal candidate are nonviable. Preferably, the growth or viability will be decreased by at least 40%. More preferably, the growth or viability will be decreased by at least 50%, 75% or at least 90% or more. Methods for measuring fungal growth and cell viability are known to those skilled in the art. By decrease in pathogenicity, is meant that the antifungal candidate causes at least a 10% decrease in the disease caused by contact of the fungal pathogen with its host, as compared to the disease caused in the absence of the antifungal candidate. Preferably, the disease will be decreased by at least 40%. More preferably, the disease will be decreased by at least 50%, 75% or at least 90% or more. Methods for measuring fungal disease are well known to those skilled in the art, and include such metrics as lesion formation, lesion size, sporulation, respiratory failure, and/or death.

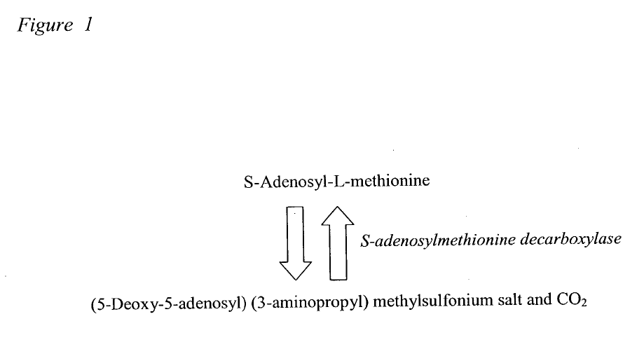

The ability of a compound to inhibit SPE2 activity can be detected using in vitro enzymatic assays in which the disappearance of a substrate or the appearance of a product is directly or indirectly detected. SPE2 catalyzes the reversible interconversion of S-adenosyl-L-methionine to (5-deoxy-5-adenosyl)(3-aminopropyl) methylsulfonium salt and CO₂ (see FIG. 1). Methods for measuring the progression of the SPE2 enzymatic reaction and/or a change in the concentration of the individual reactants S-adenosyl-L-methionine, (5-deoxy-5-adenosyl)(3-aminopropyl) methylsulfonium salt, and/or CO₂, include spectrophotometry, fluorimetry, mass spectroscopy, thin layer chromatography (TLC) and reverse phase HPLC.

Thus, the invention provides a method for identifying a test compound as a candidate for an antibiotic, comprising: contacting S-adenosyl-L-methionine with an SPE2 in the presence and absence of a test compound or contacting (5-deoxy-5-adenosyl) (3-aminopropyl) methylsulfonium salt and CO₂ with an SPE2 in the presence and absence of a test compound; and determining a change in concentration for at least one of S-adenosyl-L-methionine, (5-deoxy-5-adenosyl)(3-aminopropyl) methylsulfonium salt, and/or CO₂ in the presence and absence of the test compound, wherein a change in the concentration for any of the above reactants indicates that the test compound is a candidate for an antibiotic.

Enzymatically active fragments of M. grisea SPE2 set forth in SEQ ID NO:3 are also useful in the methods of the invention. For example, an enzymatically active polypeptide comprising at least 50 consecutive amino acid residues and at least 10% of the activity of M. grisea SPE2 set forth in SEQ ID NO:3 are useful in the methods of the invention. In addition, fragments of heterologous SPE2s are also useful in the methods of the invention. Enzymatically active polypeptides having at least 10% of the activity of SEQ ID NO:3 and at least 50%, 60%, 70%, 80%, 90%, 95%, 96%, 97%, 98% or 99% sequence identity with at least 50 consecutive amino acid residues of SEQ ID NO:3 are useful in the methods of the invention. Most preferably, the enzymatically active polypeptide has at least 50% sequence identity with at least 50 consecutive amino acid residues of SEQ ID NO:3 and at least 25%, 75% or at least 90% of the activity thereof. Thus, the invention provides a method for identifying a test compound as a candidate for an antibiotic, comprising: contacting S-adenosyl-L-methionine or (5-deoxy-5-adenosyl) (3-aminopropyl) methylsulfonium salt and CO₂ with a polypeptide selected from the group consisting of: a polypeptide consisting essentially of SEQ ID NO:3, a polypeptide having at least 50% sequence identity with the M. grisea SPE2 set forth in SEQ ID NO:3 and having at least 10% of the activity thereof, a polypeptide comprising at least 50 consecutive amino acids of M. grisea SPE2 set forth in SEQ ID NO:3 and having at least 10% of the activity thereof, and a polypeptide consisting of at least 50 amino acids and having at least 50% sequence identity with M. grisea SPE2 set forth in SEQ ID NO:3 and having at least 10% of the activity thereof, contacting S-adenosyl-L-methionine or (5-deoxy-5-adenosyl)(3-aminopropyl) methylsulfonium salt and CO₂ with the polypeptide and a test compound, and determining a change in concentration for at least one of S-adenosyl-L-methionine, (5-deoxy-5-adenosyl)(3-aminopropyl) methylsulfonium salt, and/or CO₂ in the presence and absence of the test compound, wherein a change in concentration for any of the above substances indicates that the test compound is a candidate for an antibiotic.

For the in vitro enzymatic assays, SPE2 protein and derivatives thereof may be isolated from a fungus or may be recombinantly produced in and isolated from an archael, bacterial, fungal, or other eukaryotic cell culture. Preferably these proteins are produced using an E. coli, yeast, or filamentous fungal expression system. Methods for the purification of SPE2 may be described in Yang and Cho ((1991) Biochem Biophys Res Commun 181: 1181-1186 (PMID: 1764068)). Other methods for the purification of SPE2 proteins and polypeptides are known to those skilled in the art.

As an alternative to in vitro assays, the invention also provides cell based assays. In one embodiment, the invention provides a method for identifying a test compound as a candidate for an antibiotic, comprising: a) measuring the expression or activity of an SPE2 in a cell, cells, tissue, or an organism in the absence of a test compound; b) contacting the cell, cells, tissue, or organism with the test compound and measuring the expression or activity of the SPE2 in the cell, cells, tissue, or organism; and c) comparing the expression or activity of the SPE2 in steps (a) and (b), wherein an altered expression or activity in the presence of the test compound indicates that the compound is a candidate for an antibiotic.

Expression of SPE2 can be measured by detecting the SPE2 primary transcript or mRNA, SPE2 polypeptide, or SPE2 enzymatic activity. Methods for detecting the expression of RNA and proteins are known to those skilled in the art. (Current Protocols in Molecular Biology, Ausubel et al., eds., Greene Publishing & Wiley-Interscience, New York, (1995)). The method of detection is not critical to the present invention. Methods for detecting SPE2 RNA include, but are not limited to, amplification assays such as quantitative reverse transcriptase-PCR, and/or hybridization assays such as Northern analysis, dot blots, slot blots, in-situ hybridization, transcriptional fusions using an SPE2 promoter fused to a reporter gene, DNA assays, and microarray assays.

Methods for detecting protein expression include, but are not limited to, immunodetection methods such as Western blots, ELISA assays, polyacrylamide gel electrophoresis, mass spectroscopy, and enzymatic assays. Also, any reporter gene system may be used to detect SPE2 protein expression. For detection using gene reporter systems, a polynucleotide encoding a reporter protein is fused in frame with SPE2, so as to produce a chimeric polypeptide. Methods for using reporter systems are known to those skilled in the art.

Chemicals, compounds or compositions identified by the above methods as modulators, preferably inhibitors, of SPE2 expression or activity can then be used to control fungal growth. Diseases such as rusts, mildews, and blights spread rapidly once established. Fungicides are thus routinely applied to growing and stored crops as a preventive measure, generally as foliar sprays or seed dressings. For example, compounds that inhibit fungal growth can be applied to a fungus or expressed in a fungus, in order to prevent fungal growth. Thus, the invention provides a method for inhibiting fungal growth, comprising contacting a fungus with a compound identified by the methods of the invention as having antifungal activity.

Antifungals and antifungal inhibitor candidates identified by the methods of the invention can be used to control the growth of undesired fungi, including ascomycota, zygomycota, basidiomycota, chytridiomycota, and lichens.

Examples of undesired fungi include, but are not limited to Powdery Scab (Spongospora subterranea), Grey Mould (Botrytis cinerea), White Rot (Armillaria mellea), Heartrot Fungus (Ganoderma adspersum), Brown-Rot (Piptoporus betulinus), Corn Smut (Ustilago maydis), Heartrot (Polyporus squamosus), Gray Leaf Spot (Cercospora zeae-maydis), Honey Fungus (Armillaria gallica), Root rot (Armillaria luteobubalina), Shoestring Rot (Armillaria ostoyae), Banana Anthracnose Fungus (Colletotrichum musae), Apple-rotting Fungus (Moniliniafructigena), Apple-rotting Fungus (Penicillium expansum), Clubroot Disease (Plasmodiophora brassicae), Potato Blight (Phytophthora infestans), Root pathogen (Heterobasidion annosum), Take-all Fungus (Gaeumannomyces graminis), Dutch Elm Disease (Ophiostoma ulmi), Bean Rust (Uromyces appendiculatus), Northern Leaf Spot (Cochliobolus carbonum), Milo Disease (Periconia circinata), Southern Corn Blight (Cochliobolus heterostrophus), Leaf Spot (Cochliobolus lunata), Brown Stripe (Cochliobolus stenospilus), Panama disease (Fusarium oxysporum), Wheat Head Scab Fungus (Fusarium graminearum), Cereal Foot Rot (Fusarium culmorum), Potato Black Scurf (Rhizoctonia solani), Wheat Black Stem Rust (Puccinia graminis), White mold (Sclerotinia sclerotiorum), diseases of animals such as infections of lungs, blood, brain, skin, scalp, nails or other tissues (Aspergillus fumigatus Aspergillus sp. Fusraium sp., Trichophyton sp., Epidermophyton sp., and Microsporum sp., and the like).

Also provided in the invention are methods of screening for an antibiotic by determining the in vivo activity of a test compound against two separate fungal organisms, wherein the fungal organisms comprise a first form of an SPE2 and a second form of the SPE2, respectively. In the methods of the invention, at least one of the two forms of the SPE2 has at least 10% of the activity of the polypeptide set forth in SEQ ID NO:3. The methods comprise comparing the growth of the two organisms in the presence of the test compound relative to their respective controls without the test compound. A difference in growth between the two organisms in the presence of the test compound indicates that the test compound is a candidate for an antibiotic.

Forms of an SPE2 useful in the methods of the invention are selected from the group consisting of: a nucleic acid encoding SEQ ID NO:3, a nucleic acid encoding a polypeptide consisting essentially of SEQ ID NO:3, SEQ ID NO:1 or SEQ ID NO:2, SEQ ID NO:1 or SEQ ID NO:2 comprising a mutation either reducing or abolishing SPE2 protein activity, a heterologous SPE2, and a heterologous SPE2 comprising a mutation either reducing or abolishing SPE2 protein activity. Any combination of two different forms of the SPE2 genes listed above are useful in the methods of the invention, with the caveat that at least one of the forms of the SPE2 has at least 10% of the activity of the polypeptide set forth in SEQ ID NO:3.

Thus, in one embodiment, the invention provides a method for identifying a test compound as a candidate for an antibiotic, comprising: providing an organism having a first form of an SPE2; providing an organism having a second form of the SPE2; and determining the growth of the organism having the first form of the SPE2 and the growth of the organism having the second form of the SPE2 in the presence of the test compound, wherein a difference in growth between the two organisms in the presence of the test compound indicates that the test compound is a candidate for an antibiotic. It is recognized in the art that the optional determination of the growth of the organism having the first form of the SPE2 and the growth of the organism having the second form of the SPE2 in the absence of any test compounds is performed to control for any inherent differences in growth as a result of the different genes. Growth and/or proliferation of an organism are measured by methods well known in the art such as optical density measurements, and the like. In a preferred embodiment, the organism is Magnaporthe grisea.

In another embodiment, the invention provides a method for identifying a test compound as a candidate for an antibiotic, comprising: providing an organism having a first form of an SPE2; providing a comparison organism having a second form of the SPE2; and determining the pathogenicity of the organism having the first form of the SPE2 and the organism having the second form of the SPE2 in the presence of the test compound, wherein a difference in pathogenicity between the two organisms in the presence of the test compound indicates that the test compound is a candidate for an antibiotic. In an alternate embodiment of the invention, the pathogenicity of the organism having the first form of the SPE2 and the organism having the second form of the SPE2 in the absence of any test compounds is determined to control for any inherent differences in pathogenicity as a result of the different genes. Pathogenicity of an organism is measured by methods well known in the art such as lesion number, lesion size, sporulation, and the like. In a preferred embodiment the organism is Magnaporthe grisea.

In one embodiment of the invention, the first form of an SPE2 is SEQ ID NO:1 or SEQ ID NO:2, and the second form of the SPE2 is an SPE2 that confers a growth conditional phenotype (i.e. a polyamine requiring phenotype) and/or a hypersensitivity or hyposensitivity phenotype on the organism. In a related embodiment of the invention, the second form of the SPE2 is SEQ ID NO:1 comprising a transposon insertion that reduces activity. In still another embodiment of the invention, the second form of the SPE2 is SEQ ID NO:1 comprising a transposon insertion that abolishes activity. In a related embodiment of the invention, the second form of the SPE2 is SEQ ID NO:2 comprising a transposon insertion that reduces activity. In a further embodiment of the invention, the second form of the SPE2 is SEQ ID NO:2 comprising a transposon insertion that abolishes activity.

Conditional lethal mutants and/or antipathogenic mutants identify particular biochemical and/or genetic pathways given that at least one identified target gene is present in that pathway. Knowledge of these pathways allows for the screening of test compounds as candidates for antibiotics as inhibitors of the substrates, products, proteins and/or enzymes of the pathway. The invention provides methods of screening for an antibiotic by determining whether a test compound is active against the polyamine biosynthetic pathway on which SPE2 functions. Pathways known in the art are found at the Kyoto Encyclopedia of Genes and Genomes and in standard biochemistry texts (See, e.g. Lehninger et al., Principles of Biochemistry, New York, Worth Publishers (1993)).

Thus, in one embodiment, the invention provides a method for screening for test compounds acting against the biochemical and/or genetic pathway or pathways in which SPE2 functions, comprising: providing an organism having a first form of a gene in the polyamine biosynthetic pathway; providing an organism having a second form of the gene in the polyamine biosynthetic pathway; and determining the growth of the two organisms in the presence of a test compound, wherein a difference in growth between the organism having the first form of the gene and the organism having the second form of the gene in the presence of the test compound indicates that the test compound is a candidate for an antibiotic. It is recognized in the art that the optional determination of the growth of the organism having the first form of the gene and the organism having the second form of the gene in the absence of any test compounds is performed to control for any inherent differences in growth as a result of the different genes. Growth and/or proliferation of an organism are measured by methods well known in the art, such as optical density measurements and the like. In a preferred embodiment, the organism is Magnaporthe grisea.

The forms of a gene in the polyamine biosynthetic pathway useful in the methods of the invention include, for example, wild-type and mutated genes encoding putrescine aminopropyltransferase and S-adenosylmethionine decarboxylase from any organism, preferably from a fungal organism, and most preferrably from M. grisea. The forms of a mutated gene in the polyamine biosynthetic pathway comprise a mutation either reducing or abolishing protein activity. In one example, the form of a gene in the polyamine biosynthetic pathway comprises a transposon insertion. Any combination of a first form of a gene in the polyamine biosynthetic pathway and a second form of the gene listed above are useful in the methods of the invention, with the limitation that one of the forms of a gene in the polyamine biosynthetic pathway has at least 10% of the activity of the corresponding M. grisea gene.

In another embodiment, the invention provides a method for screening for test compounds acting against the biochemical and/or genetic pathway or pathways in which SPE2 functions, comprising: providing an organism having a first form of a gene in the polyamine biosynthetic pathway; providing an organism having a second form of the gene in the polyamine biosynthetic pathway; and determining the pathogenicity of the two organisms in the presence of the test compound, wherein a difference in pathogenicity between the organism having the first form of the gene and the organism having the second form of the gene in the presence of the test compound indicates that the test compound is a candidate for an antibiotic. In an optional embodiment of the invention, the pathogenicity of the two organisms in the absence of any test compounds is determined to control for any inherent differences in pathogenicity as a result of the different genes. Pathogenicity of an organism is measured by methods well known in the art such as lesion number, lesion size, sporulation, and the like. In a preferred embodiment the organism is Magnaporthe grisea.

Thus, in an alternate embodiment, the invention provides a method for screening for test compounds acting against the biochemical and/or genetic pathway or pathways in which SPE2 functions, comprising: providing paired growth media containing a test compound, wherein the paired growth media comprise a first medium and a second medium and the second medium contains a higher level of polyamine than the first medium; inoculating the first and the second medium with an organism; and determining the growth of the organism, wherein a difference in growth of the organism between the first and the second medium indicates that the test compound is a candidate for an antibiotic. In one embodiment of the invention, the growth of the organism is determined in the first and the second medium in the absence of any test compounds to control for any inherent differences in growth as a result of the different media. Growth and/or proliferation of the organism are measured by methods well known in the art such as optical density measurements, and the like. In a preferred embodiment, the organism is Magnaporthe grisea.

One embodiment of the invention is directed to the use of multi-well plates for screening of antibiotic compounds. The use of multi-well plates is a format that readily accommodates multiple different assays to characterize various compounds, concentrations of compounds, and fungal organisms in varying combinations and formats. Certain testing parameters for the screening method can significantly affect the identification of growth inhibitors, and thus can be manipulated to optimize screening efficiency and/or reliability. Notable among these factors are variable sensitivities of different mutants, increasing hypersensitivity with increasingly less permissive conditions, an apparent increase in hypersensitivity with increasing compound concentration, and other factors known to those in the art.

### EXPERIMENTAL

### EXAMPLE 1

### Construction of Plasmids with a Transposon Containing a Selectable Marker

Construction of Sif transposon: Sif was constructed using the GPS3 vector from the GPS-M mutagenesis system from New England Biolabs, Inc. (Beverly, Mass.) as a backbone. This system is based on the bacterial transposon Tn7. The following manipulations were done to GPS3 according to Sambrook et al. (1989) Molecular Cloning, a Laboratory Manual, Cold Spring Harbor Laboratory Press. The kanamycin resistance gene (npt) contained between the Tn7 arms was removed by EcoRV digestion. The bacterial hygromycin B phosphotransferase (hph) gene (Gritz and Davies (1983) Gene 25: 179-88 (PMID: 6319235)) under control of the Aspergillus nidulans trpC promoter and terminator (Mullaney et al. (1985) Mol Gen Genet 199: 37-45 (PMID: 3158796)) was cloned by a HpaI/EcoRV blunt ligation into the Tn7 arms of the GPS3 vector yielding pSif1 Excision of the ampicillin resistance gene (bla) from pSif1 was achieved by cutting pSif1 with XmnI and BglI followed by a T4 DNA polymerase treatment to remove the 3′ overhangs left by the BglI digestion and religation of the plasmid to yield pSif. Top 10F′ electrocompetent E. coli cells (Invitrogen) were transformed with ligation mixture according to manufacturer's recommendations. Transformants containing the Sif transposon were selected on LB agar (Sambrook et al. (1989) Molecular Cloning, a Laboratory Manual) containing 50 ug/ml of hygromycin B (Sigma Chem. Co., St. Louis, Mo.).

### EXAMPLE 2

### Construction of a Fungal Cosmid Library

Cosmid libraries were constructed in the pcosKA5 vector (Hamer et al. (2001) Proc Natl Acad Sci USA 98: 5110-15 (PMID: 11296265)) as described in Sambrook et al. (1989) Molecular Cloning, a Laboratory Manual. Cosmid libraries were quality checked by pulsed-field gel electrophoresis, restriction digestion analysis, and PCR identification of single genes.

### EXAMPLE 3

### Construction of Cosmids with Transposon Insertion into Fungal Genes

Sif Transposition into a Cosmid: Transposition of Sif into the cosmid framework was carried out as described by the GPS-M mutagenesis system (New England Biolabs, Inc.). Briefly, 2 ul of the 10× GPS buffer, 70 ng of supercoiled pSIF, 8-12 ug of target cosmid DNA were mixed and taken to a final volume of 20 ul with water. 1 ul of transposase (TnsABC) was added to the reaction and incubated for 10 minutes at 37° C. to allow the assembly reaction to happen. After the assembly reaction, 1 ul of start solution was added to the tube, mixed well and incubated for 1 hour at 37° C. followed by heat inactivation of the proteins at 75° C. for 10 min. Destruction of the remaining untransposed pSif was done by PISceI digestion at 37° C. for 2 hours followed by 10 min incubation at 75° C. to inactivate the proteins. Transformation of Top 10F′ electrocompetent cells (Invitrogen) was done according to manufacturers recommendations. Sif-containing cosmid transformants were selected by growth on LB agar plates containing 50 ug/ml of hygromycin B (Sigma Chem. Co.) and 100 ug/ml of Ampicillin (Sigma Chem. Co.).

### EXAMPLE 4

### High Throughput Preparation and Verification of Transposon Insertion into the M. grisea SPE2 Gene

E. coli strains containing cosmids with transposon insertions were picked to 96 well growth blocks (Beckman Co.) containing 1.5 ml of TB (Terrific Broth, Sambrook et al. (1989) Molecular Cloning, a Laboratory Manual, Cold Spring Harbor Laboratory Press) supplemented with 50 ug/ml of ampicillin. Blocks were incubated with shaking at 37° C. overnight. E. coli cells were pelleted by centrifugation and cosmids were isolated by a modified alkaline lysis method (Marra et al. (1997) Genome Res 7: 1072-84 (PMID: 9371743)). DNA quality was checked by electrophoresis on agarose gels. Cosmids were sequenced using primers from the ends of each transposon and commercial dideoxy sequencing kits (Big Dye Terminators, Perkin Elmer Co.). Sequencing reactions were analyzed on an ABI377 DNA sequencer (Perkin Elmer Co.).

DNA sequences adjacent to the site of the insertion were collected and used to search DNA and protein databases using the BLAST algorithms (Altschul et al. (1997) Nucleic Acids Res 25: 3389-3402 (PMID: 9254694)). A single insertion of SIF into the Magnaporthe grisea SPE2 gene was chosen for further analysis. This construct was designated cpgmra002300c08 and it contains the SIF transposon in the coding region relative to of the Saccharomyces cerevisiae homologue (total length: 396 amino acids, GENBANK: 6324521).

### EXAMPLE 5

### Preparation of SPE2 Cosmid DNA and Transformation of Magnaporthe grisea

Cosmid DNA from the SPE2 transposon tagged cosmid clone was prepared using QIAGEN Plasmid Maxi Kit (QIAGEN), and digested by PI-PspI (New England Biolabs, Inc.). Fungal electro-transformation was performed essentially as described (Wu et al. (1997) MPMI 10: 700-708). Briefly, M. grisea strain Guy 11 was grown in complete liquid media (Talbot et al. (1993) Plant Cell 5: 1575-1590 (PMID: 8312740)) shaking at 120 rpm for 3 days at 25° C. in the dark. Mycelia was harvested and washed with sterile H₂O and digested with 4 mg/ml beta-glucanase (InterSpex) for 4-6 hours to generate protoplasts. Protoplasts were collected by centrifugation and resuspended in 20% sucrose at the concentration of 2×10⁸ protoplasts/ml. 50 ul protoplast suspension was mixed with 10-20 ug of the cosmid DNA and pulsed using Gene Pulser II (BioRad) set with the following parameters: resistance 200 ohm, capacitance 25uF, voltage 0.6 kV. Transformed protoplasts were regenerated in complete agar media (CM, Talbot et al. (1993) Plant Cell 5: 1575-1590 (PMID: 8312740)) with the addition of 20% sucrose for one day, then overlayed with CM agar media containing hygromycin B (250 ug/ml) to select transformants. Transformants were screened for homologous recombination events in the target gene by PCR (Hamer et al. (2001) Proc Natl Acad Sci USA 98: 5110-15 (PMID: 11296265)). Two independent strains were identified and are hereby referred to as KO1-1 and KO1-36, respectively.

### EXAMPLE 6

### Effect of Transposon Insertion on Magnaporthe Pathogenicity

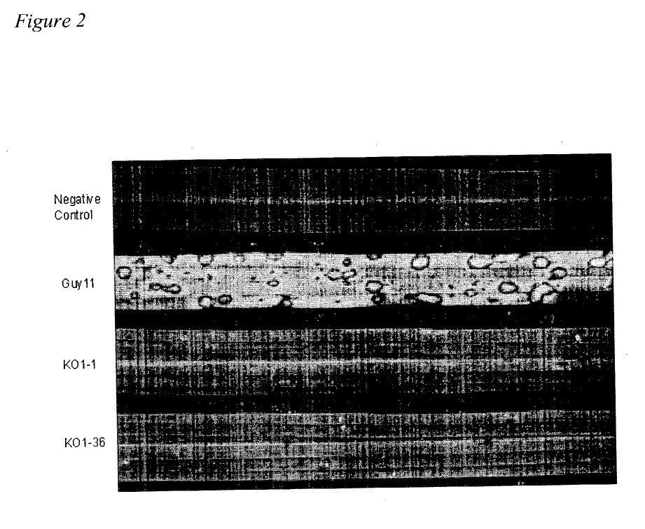

The target fungal strains, KO1-1 and KO1-36, obtained in Example 5 and the wild type strain, Guy11, were subjected to a pathogenicity assay to observe infection over a 1-week period. Rice infection assays were performed using Indian rice cultivar CO39 essentially as described in Valent et al. ((1991) Genetics 127: 87-101 (PMID: 2016048)). All three strains were grown for spore production on complete agar media. Spores were harvested and the concentration of spores adjusted for whole plant inoculations. Two-week-old seedlings of cultivar CO39 were sprayed with 12 ml of conidial suspension (5×10⁴ conidia per ml in 0.01% TWEEN-20 (Polyoxyethylensorbitan monolaureate) solution). The inoculated plants were incubated in a dew chamber at 27° C. in the dark for 36 hours, and transferred to a growth chamber (27° C. 12 hours/21° C. 12 hours 70% humidity) for an additional 5.5 days. Leaf samples were taken at 3, 5, and 7 days post-inoculation and examined for signs of successful infection (i.e. lesions). FIG. 2 shows the effects of SPE2 gene disruption on Magnaporthe infection at five days post-inoculation.

### EXAMPLE 7

### Cloning and Expression Strategies, Extraction and Purification of S-adenosylmethionine decarboxylase Protein

The following protocol may be employed to obtain a isolated S-adenosylmethionine decarboxylase protein.

Cloning and Expression Strategies:

An SPE2 cDNA gene can be cloned into E. coli (pET vectors-Novagen), Baculovirus (Pharmingen) and Yeast (Invitrogen) expression vectors containing His/fusion protein tags, and the expression of recombinant protein can be evaluated by SDS-PAGE and Western blot analysis.

Extraction:

Extract recombinant protein from 250 ml cell pellet in 3 ml of extraction buffer by sonicating 6 times, with 6 sec pulses at 4° C. Centrifuge extract at 15000×g for 10 min and collect supernatant. Assess biological activity of the recombinant protein by activity assay.

Purification:

Purify recombinant protein by Ni-NTA affinity chromatography. Purification protocol: perform all steps at 4° C.:

Use 3 ml Ni-beads (Qiagen)

Equilibrate column with the buffer

Load protein extract

Wash with the equilibration buffer

Elute bound protein with 0.5 M imidazole

### EXAMPLE 8

### Assays for Testing Binding of Test Compounds to S-adenosylmethionine decarboxylase

The following protocol may be employed to identify test compounds that bind to the S-adenosylmethionine decarboxylase protein.

Isolated full-length S-adenosylmethionine decarboxylase polypeptide with a His/fusion protein tag (Example 7) is bound to a HISGRAB Nickel Coated Plate (Pierce, Rockford, Ill.) following manufacturer's instructions.

Buffer conditions are optimized (e.g. ionic strength or pH, Kinch et al. (1999) Mol Biochem Parasitol 101: 1-11 (PMID: 10413038)) for binding of radiolabeled S-Adenosyl-L-methionine (Sigma-Aldritch) to the bound S-adenosylmethionine decarboxylase.

Screening of test compounds is performed by adding test compound and S-Adenosyl-L-methoinine (Sigma-Aldritch) to the wells of the HISGRAB plate containing bound S-adenosylmethionine decarboxylase.

The wells are washed to remove excess labeled ligand and scintillation fluid (SCINTIVERSE, Fisher Scientific) is added to each well.

The plates are read in a microplate scintillation counter.

Candidate compounds are identified as wells with lower radioactivity as compared to control wells with no test compound added.

Additionally, an isolated polypeptide comprising 10-50 amino acids from the M. grisea S-adenosylmethionine decarboxylase is screened in the same way. A polypeptide comprising 10-50 amino acids is generated by subcloning a portion of the SPE2 gene into a protein expression vector that adds a His-Tag when expressed (see Example 7). Oligonucleotide primers are designed to amplify a portion of the SPE2 gene using the polymerase chain reaction amplification method. The DNA fragment encoding a polypeptide of 10-50 amino acids is cloned into an expression vector, expressed in a host organism and isolated as described in Example 7 above.

Test compounds that bind SPE2 are further tested for antibiotic activity. M. grisea is grown as described for spore production on oatmeal agar media (Talbot et al. (1993) Plant Cell 5: 1575-1590 (PMID: 8312740)). Spores are harvested into minimal media (Talbot et al. (1993) Plant Cell 5: 1575-1590 (PMID: 8312740)) to a concentration of 2×10⁵ spores/ml and the culture is divided. The test compound is added to one culture to a final concentration of 20-100 μg/ml. Solvent only is added to the second culture. The plates are incubated at 25° C. for seven days and optical density measurements at 590 nm are taken daily. The growth curves of the solvent control sample and the test compound sample are compared. A test compound is an antibiotic candidate if the growth of the culture containing the test compound is less than the growth of the control culture.

### EXAMPLE 9

### Assays for Testing Inhibitors or Candidates for Inhibition of S-adenosylmethionine decarboxylase Activity

The enzymatic activity of S-adenosylmethionine decarboxylase is determined in the presence and absence of candidate compounds in a suitable reaction mixture, such as described by Kinch et al. ((1999) Mol Biochem Parasitol 101: 1-11 (PMID: 10413038)). Candidate compounds are identified when a decrease in products or a lack of decrease in substrates is detected with the reaction proceeding in either direction.

Additionally, the enzymatic activity of a polypeptide comprising 10-50 amino acids from the M. grisea S-adenosylmethionine decarboxylase is determined in the presence and absence of candidate compounds in a suitable reaction mixture, such as described by Kinch et al. ((1999) Mol Biochem Parasitol 101: 1-11 (PMID: 10413038)). A polypeptide comprising 10-50 amino acids is generated by subcloning a portion of the SPE2 gene into a protein expression vector that adds a His-Tag when expressed (see Example 7). Oligonucleotide primers are designed to amplify a portion of the SPE2 gene using polymerase chain reaction amplification method. The DNA fragment encoding a polypeptide of 10-50 amino acids is cloned into an expression vector, expressed and isolated as described in Example 7 above.

Test compounds identified as inhibitors of SPE2 activity are further tested for antibiotic activity. Magnaporthe grisea fungal cells are grown under standard fungal growth conditions that are well known and described in the art. M. grisea is grown as described for spore production on oatmeal agar media (Talbot et al. (1993) Plant Cell 5: 1575-1590 (PMID: 8312740)). Spores are harvested into minimal media (Talbot et al. (1993) Plant Cell 5: 1575-1590 (PMID: 8312740)) to a concentration of 2×10⁵ spores/ml and the culture is divided. The test compound is added to one culture to a final concentration of 20-100 μg/ml. Solvent only is added to the second culture. The plates are incubated at 25° C. for seven days and optical density measurements at 590 nm are taken daily. The growth curves of the solvent control sample and the test compound sample are compared. A test compound is an antibiotic candidate if the growth of the culture containing the test compound is less than the growth of the control culture.

### EXAMPLE 10

### Assays for Testing Compounds for Alteration of S-adenosylmethionine decarboxylase Gene Expression

Magnaporthe grisea fungal cells are grown under standard fungal growth conditions that are well known and described in the art. Wild-type M. grisea spores are harvested from cultures grown on complete agar or oatmeal agar media after growth for 10-13 days in the light at 25° C. using a moistened cotton swab. The concentration of spores is determined using a hemacytometer and spore suspensions are prepared in a minimal growth medium to a concentration of 2×10⁵ spores per ml. 25 ml cultures are prepared to which test compounds will be added at various concentrations. A culture with no test compound present is included as a control. The cultures are incubated at 25° C. for 3 days after which test compound or solvent only control is added. The cultures are incubated an additional 18 hours. Fungal mycelia is harvested by filtration through Miracloth (CalBiochem, La Jolla, Calif.), washed with water and frozen in liquid nitrogen. Total RNA is extracted with TRIZOL Reagent using the methods provided by the manufacturer (Life Technologies, Rockville, Md.). Expression is analyzed by Northern analysis of the RNA samples as described (Sambrook et al. (1989) Molecular Cloning, a Laboratory Manual, Cold Spring Harbor Laboratory Press) using a radiolabeled fragment of the SPE2 gene as a probe. Test compounds resulting in a reduced level of SPE2 mRNA relative to the untreated control sample are identified as candidate antibiotic compounds.

### EXAMPLE 11

### In Vivo Cell Based Assay Screening Protocol with a Fungal Strain Containing a Mutant Form of S-adenosylmethionine decarboxylase with No Activity

Magnaporthe grisea fungal cells containing a mutant form of the SPE2 gene which abolishes enzyme activity, such as a gene containing a transposon insertion (see Examples 4 and 5), are grown under standard fungal growth conditions that are well known and described in the art. Magnaporthe grisea spores are harvested from cultures grown on complete agar medium containing 4 1 mM polyamine spermidine (Sigma-Aldrich Co.) after growth for 10-13 days in the light at 25° C. using a moistened cotton swab. The concentration of spores is determined using a hemacytometer and spore suspensions are prepared in a minimal growth medium containing 100 μM polyamine spermidine to a concentration of 2×10⁵ spores per ml. Approximately 4×10⁴ spores are added to each well of 96-well plates to which a test compound is added (at varying concentrations). The total volume in each well is 200 μl. Wells with no test compound present (growth control), and wells without cells are included as controls (negative control). The plates are incubated at 25° C. for seven days and optical density measurements at 590 nm are taken daily. Wild type cells are screened under the same conditions. The effect of each compound on the mutant and wild-type fungal strains is measured against the growth control and the percent of inhibition is calculated as the OD₅₉₀ (fungal strain plus test compound)/OD₅₉₀ (growth control)×100. The percent of growth inhibition as a result of a test compound on a fungal strain and that on the wild type cells are compared. Compounds that show differential growth inhibition between the mutant and the wild type are identified as potential antifungal compounds. Similar protocols may be found in Kirsch and DiDomenico ((1994) Biotechnology 26: 177-221 (PMID: 7749303)).

### EXAMPLE 12

### In Vivo Cell Based Assay Screening Protocol with a Fungal Strain Containing a Mutant Form of S-adenosylmethionine decarboxylase with Reduced Activity

Magnaporthe grisea fungal cells containing a mutant form of the SPE2 gene, such as a promoter truncation that reduces expression, are grown under standard fungal growth conditions that are well known and described in the art. A promoter truncation is made by deleting a portion of the promoter upstream of the transcription start site using standard molecular biology techniques that are well known and described in the art (Sambrook et al. (1989) Molecular Cloning, a Laboratory Manual, Cold Spring Harbor Laboratory Press). Magnaporthe grisea spores are harvested from cultures grown on complete agar medium containing 4 1mM polyamine spermidine (Sigma-Aldrich Co.) after growth for 10-13 days in the light at 25° C. using a moistened cotton swab. The concentration of spores is determined using a hemacytometer and spore suspensions are prepared in a minimal growth medium to a concentration of 2×10⁵ spores per ml. Approximately 4×10⁴ spores are added to each well of 96-well plates to which a test compound is added (at varying concentrations). The total volume in each well is 200111. Wells with no test compound present (growth control), and wells without cells are included as controls (negative control). The plates are incubated at 25° C. for seven days and optical density measurements at 590 nm are taken daily. Wild type cells are screened under the same conditions. The effect of each compound on the mutant and wild-type fungal strains is measured against the growth control and the percent of inhibition is calculated as the OD₅₉₀ (fungal strain plus test compound)/OD₅₉₀ (growth control)×100. The percent of growth inhibition as a result of a test compound on a fungal strain and that on the wild-type cells are compared. Compounds that show differential growth inhibition between the mutant and the wild type are identified as potential antifungal compounds. Similar protocols may be found in Kirsch and DiDomenico ((1994) Biotechnology 26: 177-221).

### EXAMPLE 13

### In Vivo Cell Based Assay Screening Protocol with a Fungal Strain Containing a Mutant Form of a Polyamine Biosynthetic Gene with No Activity

Magnaporthe grisea fungal cells containing a mutant form of a gene in the polyamine biosynthetic pathway (e.g. putrescine aminopropyltransferase, ornithine decarboxylase, or spermine synthase) are grown under standard fungal growth conditions that are well known and described in the art. Magnaporthe grisea spores are harvested from cultures grown on complete agar medium containing 4 1mM spermidinepolyamine (Sigma-Aldrich Co.) after growth for 10-13 days in the light at 25° C. using a moistened cotton swab. The concentration of spores is determined using a hemacytometer and spore suspensions are prepared in a minimal growth medium containing 100 μM spermidinepolyamine to a concentration of 2×10⁵ spores per ml. Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for seven days and optical density measurements at 590 nm are taken daily. Wild type cells are screened under the same conditions. The effect of each compound on the mutant and wild-type fungal strains is measured against the growth control and the percent of inhibition is calculated as the OD₅₉₀ (fungal strain plus test compound)/OD₅₉₀ (growth control)×100. The percent of growth inhibition as a result of a test compound on a fungal strain and that on the wild type cells are compared. Compounds that show differential growth inhibition between the mutant and the wild-type are identified as potential antifungal compounds. Similar protocols may be found in Kirsch and DiDomenico ((1994) Biotechnology 26: 177-221).

### EXAMPLE 14

### In Vivo Cell Based Assay Screening Protocol with a Fungal Strain Containing a Mutant Form of a Polyamine Biosynthetic Gene with Reduced Activity

Magnaporthe grisea fungal cells containing a mutant form of a gene in the polyamine biosynthetic pathway ((e.g. putrescine aminopropyltransferase, ornithine decarboxylase, or spermine synthase), such as a promoter truncation that reduces expression, are grown under standard fungal growth conditions that are well known and described in the art. A promoter truncation is made by deleting a portion of the promoter upstream of the transcription start site using standard molecular biology techniques that are well known and described in the art (Sambrook et al. (1989) Molecular Cloning, a Laboratory Manual, Cold Spring Harbor Laboratory Press). Magnaporthe grisea fungal cells containing a mutant form are grown under standard fungal growth conditions that are well known and described in the art. Magnaporthe grisea spores are harvested from cultures grown on complete agar medium containing 4 1 mM spermidinepolyamine (Sigma-Aldrich Co.) after growth for 10-13 days in the light at 25° C. using a moistened cotton swab. The concentration of spores is determined using a hemacytometer and spore suspensions are prepared in a minimal growth medium to a concentration of 2×10⁵ spores per ml. Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for seven days and optical density measurements at 590nm are taken daily. Wild type cells are screened under the same conditions. The effect of each compound on the mutant and wild-type fungal strains is measured against the growth control and the percent of inhibition is calculated as the OD₅₉₀ (fungal strain plus test compound)/OD₅₉₀ (growth control)×100. The percent of growth inhibition as a result of a test compound on a fungal strain and that on the wild type cells are compared. Compounds that show differential growth inhibition between the mutant and the wild type are identified as potential antifungal compounds. Similar protocols may be found in Kirsch and DiDomenico ((1994) Biotechnology 26: 177-221).

### EXAMPLE 15

### In Vivo Cell Based Assay Screening Protocol with a Fungal Strain Containing a Fungal SPE2 and a Second Fungal Strain Containing a Heterologous SPE2 Gene

Wild-type Magnaporthe grisea fungal cells and M. grisea fungal cells lacking a functional SPE2 gene and containing a S-adenosylmethionine decarboxylase gene from Neurospora crassa (Genbank 4929540, 63% sequence identity) are grown under standard fungal growth conditions that are well known and described in the art. An M. grisea strain carrying a heterologous SPE2 gene is made as follows:

An M. grisea strain is made with a nonfunctional SPE2 gene, such as one containing a transposon insertion in the native gene (see Examples 4 and 5).

A construct containing a heterologous SPE2 gene is made by cloning the S-adenosylmethionine decarboxylase gene from Neurospora crassa into a fungal expression vector containing a trpC promoter and terminator (e.g. pCB1003, Carroll et al. (1994) Fungal Gen News Lett 41: 22) using standard molecular biology techniques that are well known and described in the art (Sambrook et al. (1989) Molecular Cloning, a Laboratory Manual).

The said construct is used to transform the M. grisea strain lacking a functional SPE2 gene (see Example 5). Transformants are selected on minimal agar medium lacking spermidinepolyamine. Only transformants carrying a functional SPE2 gene will grow.

Wild-type strains of Magnaporthe grisea and strains containing a heterologous form of SPE2 are grown under standard fungal growth conditions that are well known and described in the art. Magnaporthe grisea spores are harvested from cultures grown on complete agar medium after growth for 10-13 days in the light at 25° C. using a moistened cotton swab. The concentration of spores is determined using a hemacytometer and spore suspensions are prepared in a minimal growth medium to a concentration of 2×10⁵ spores per ml. Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for seven days and optical density measurements at 590 nm are taken daily. The effect of each compound on the wild-type and heterologous fungal strains is measured against the growth control and the percent of inhibition is calculated as the OD₅₉₀ (fungal strain plus test compound)/OD₅₉₀ (growth control)×100. The percent of growth inhibition as a result of a test compound on the wild-type and heterologous fungal strains are compared. Compounds that show differential growth inhibition between the wild-type and heterologous strains are identified as potential antifungal compounds with specificity to the native or heterologous SPE2 gene products. Similar protocols may be found in Kirsch and DiDomenico ((1994) Biotechnology 26: 177-221).

### EXAMPLE 16

### Pathway Specific In Vivo Assay Screening Protocol

Magnaporthe grisea fungal cells are grown under standard fungal growth conditions that are well known and described in the art. Wild-type M. grisea spores are harvested from cultures grown on oatmeal agar media after growth for 10-13 days in the light at 25° C. using a moistened cotton swab. The concentration of spores is determined using a hemocytometer and spore suspensions are prepared in a minimal growth medium and a minimal growth medium containing 4 1 mM spermidinepolyamine (Sigma-Aldrich Co.) to a concentration of 2×10⁵ spores per ml. The minimal growth media contains carbon, nitrogen, phosphate, and sulfate sources, and magnesium, calcium, and trace elements (for example, see inoculating fluid in Example 7). Spore suspensions are added to each well of a 96-well microtiter plate (approximately 4×10⁴ spores/well). For each well containing a spore suspension in minimal media, an additional well is present containing a spore suspension in minimal medium containing 4 1 mM spermidinepolyamine. Test compounds are added to wells containing spores in minimal media and minimal media containing spermidinepolyamine. The total volume in each well is 200 μl. Both minimal media and spermidinepolyamine containing media wells with no test compound are provided as controls. The plates are incubated at 25° C. for seven days and optical density measurements at 590 nm are taken daily. A compound is identified as a candidate for an antibiotic acting against the polyamine biosynthetic pathway when the observed growth in the well containing minimal media is less than the observed growth in the well containing spermidinepolyamine as a result of the addition of the test compound. Similar protocols may be found in Kirsch and DiDomenico ((1994) Biotechnology 26: 177-221).

While the foregoing describes certain embodiments of the invention, it will be understood by those skilled in the art that variations and modifications may be made and still fall within the scope of the invention. The foregoing examples are intended to exemplify various specific embodiments of the invention and do not limit its scope in any manner.

What is claimed is:

1. A method for identifying a test compound as a candidate for an antibiotic, comprising:
a) contacting an S-adenosylmethionine decarboxylase polypeptide with a test compound; and
b) detecting the presence or absence of binding between the test compound and the S-adenosylmethionine decarboxylase polypeptide, wherein binding indicates that the test compound is a candidate for an antibiotic.

2. The method of claim 1, wherein the S-adenosylmethionine decarboxylase polypeptide is a fungal S-adenosylmethionine decarboxylase polypeptide.

3. The method of claim 1, wherein the S-adenosylmethionine decarboxylase polypeptide is a Magnaporthe S-adenosylmethionine decarboxylase polypeptide.

4. The method of claim 1, wherein the S-adenosylmethionine decarboxylase polypeptide is SEQ ID NO:3.

5. A method for identifying a test compound as a candidate for an antibiotic, comprising:
a) contacting a test compound with a polypeptide selected from the group consisting of:
i) a polypeptide consisting essentially of SEQ ID NO:3;
ii) a polypeptide having at least ten consecutive amino acids of SEQ ID NO:3;
iii) a polypeptide having at least 50% sequence identity with SEQ ID NO:3 and at least 10% of the activity of SEQ ID NO:3; and
iv) a polypeptide consisting of at least 50 amino acids having at least 50% sequence identity with SEQ ID NO:3 and at least 10% of the activity of SEQ ID NO:3; and
b) detecting the presence and/or absence of binding between the test compound and the polypeptide, wherein binding indicates that the test compound is a candidate for an antibiotic.

6. A method for identifying a test compound as a candidate for an antibiotic, comprising:
a) contacting S-adenosyl-L-methionine with an S-adenosylmethionine decarboxylase in the presence and absence of a test compound or contacting (5-deoxy-5-adenosyl) (3-aminopropyl) methylsulfonium salt, and CO₂ with an S-adenosylmethionine decarboxylase in the presence and absence of a test compound; and
b) determining a change in concentration for at least one of S-adenosyl-L-methionine, (5-deoxy-5-adenosyl) (3-aminopropyl) methylsulfonium salt, and/or CO₂ in the presence and absence of the test compound, wherein a change in the concentration for any of S-adenosyl-L-methionine, (5-deoxy-5-adenosyl) (3-aminopropyl) methylsulfonium salt, and/or CO₂indicates that the test compound is a candidate for an antibiotic.

7. The method of claim 6, wherein the S-adenosylmethionine decarboxylase is a fungal S-adenosylmethionine decarboxylase.

8. The method of claim 7, wherein the S-adenosylmethionine decarboxylase is a Magnaporthe S-adenosylmethionine decarboxylase.

9. The method of claim 8, wherein the S-adenosylmethionine decarboxylase is SEQ ID NO:3.

10. A method for identifying a test compound as a candidate for an antibiotic, comprising:
a) contacting an S-adenosylmethionine decarboxylase polypeptide with S-adenosyl-L-methionine in the presence and absence of a test compound or with (5-deoxy-5-adenosyl) (3-aminopropyl) methylsulfonium salt, and CO₂ in the presence and absence of a test compound, wherein the S-adenosylmethionine decarboxylase polypeptide is selected from the group consisting of:
i) a polypeptide having at least 50% sequence identity with SEQ ID NO:3 and at least 10% of the activity of SEQ ID NO:3,
ii) a polypeptide consisting essentially of SEQ ID NO:3,
iii) a polypeptide comprising at least 50 consecutive amino acids of SEQ ID NO:3 and having at least 10% of the activity of SEQ ID NO:3; and
iv) a polypeptide consisting of at least 50 amino acids having at least 50% sequence identity with SEQ ID NO:3 and having at least 10% of the activity of SEQ ID NO:3; and
b) determining a change in concentration for at least one of S-adenosyl-L-methionine, (5-deoxy-5-adenosyl) (3-aminopropyl) methylsulfonium salt, and/or CO₂ in the presence and absence of the test compound, wherein a change in the concentration for any of S-adenosyl-L-methionine, (5-deoxy-5-adenosyl) (3-aminopropyl) methylsulfonium salt, and/or CO₂ indicates that the test compound is a candidate for an antibiotic.

11. A method for identifying a test compound as a candidate for an antibiotic, comprising:
a) measuring the expression of an S-adenosylmethionine decarboxylase in an organism, or a cell or tissue thereof, in the presence and absence of a test compound; and
b) comparing the expression of the S-adenosylmethionine decarboxylase in the presence and absence of the test compound, wherein an altered expression in the presence of the test compound indicates that the test compound is a candidate for an antibiotic.

12. The method of claim 11, wherein the organism is a fungus.

13. The method of claim 12, wherein the organism is Magnaporthe.

14. The method of claim 11, wherein the S-adenosylmethionine decarboxylase is SEQ ID NO:3.

15. The method of claim 11, wherein the expression of the S-adenosylmethionine decarboxylase is measured by detecting the S-adenosylmethionine decarboxylase mRNA.

16. The method of claim 11, wherein the expression of the S-adenosylmethionine decarboxylase is measured by detecting the S-adenosylmethionine decarboxylase polypeptide.

17. The method of claim 11, wherein the expression of the S-adenosylmethionine decarboxylase is measured by detecting the S-adenosylmethionine decarboxylase polypeptide enzyme activity.

18. A method for identifying a test compound as a candidate for an antibiotic, comprising:
a) providing a fungal organism having a first form of an S-adenosylmethionine decarboxylase;
b) providing a fungal organism having a second form of the S-adenosylmethionine decarboxylase, wherein one of the first or the second form of the S-adenosylmethionine decarboxylase has at least 10% of the activity of SEQ ID NO:3; and
c) determining the growth of the organism having the first form of the S-adenosylmethionine decarboxylase and the organism having the second form of the S-adenosylmethionine decarboxylase in the presence of a test compound,
wherein a difference in growth between the two organisms in the presence of the test compound indicates that the test compound is a candidate for an antibiotic.

19. The method of claim 18, wherein the fungal organism having the first form of the S-adensoylmethionine decarboxylase and the fungal organism having the second form of the S-adenosylmethionine decarboxylase are Magnaporthe and the first and the second form of the S-adenosylmethionine decarboxylase are fungal S-adenosylmethionine decarboxylase s.

20. The method of claim 18, wherein the first form of the S-adenosylmethionine decarboxylase is SEQ ID NO:1 or SEQ ID NO:2.

21. The method of claim 18, wherein the fungal organism having the first form of the S-adensoylmethionine decarboxylase and the fungal organism having the second form of the S-adenosylmethionine decarboxylase are Magnaporthe and the first form of the S-adenosylmethionine decarboxylase is SEQ ID NO:1 or SEQ ID NO:2.

22. The method of claim 18, wherein the fungal organism having the first form of the S-adensoylmethionine decarboxylase and the fungal organism having the second form of the S-adenosylmethionine decarboxylase are Magnaporthe, the first form of the S-adensoylmethionine decarboxylase is SEQ ID NO:1 or SEQ ID NO:2, and the second form of the S-adenosylmethionine decarboxylase is a heterologous S-adenosylmethionine decarboxylase.

23. The method of claim 18, wherein the fungal organism having the first form of the S-adenosylmethionine decarboxylase and the fungal organism having the second form of the S-adenosylmethionine decarboxylase are Magnaporthe, the first form of the S-adensoylmethionine decarboxylase is SEQ ID NO:1 or SEQ ID NO:2, and the second form of the S-adenosylmethionine decarboxylase is SEQ ID NO:1 or SEQ ID NO:2 comprising a transposon insertion that reduces or abolishes S-adenosylmethionine decarboxylase activity.

24. A method for identifying a test compound as a candidate for an antibiotic, comprising:
a) providing a fungal organism having a first form of an S-adenosylmethionine decarboxylase;
b) providing a fungal organism having a second form of the S-adenosylmethionine decarboxylase, wherein one of the first or the second form of the S-adenosylmethionine decarboxylase has at least 10% of the activity of SEQ ID NO:3; and
c) determining the pathogenicity of the organism having the first form of the S-adensoylmethionine decarboxylase and the organism having the second form of the S-adenosylmethionine decarboxylase in the presence of a test compound,
wherein a difference in pathogenicity between the two organisms in the presence of the test compound indicates that the test compound is a candidate for an antibiotic.

25. The method of claim 24, wherein the fungal organism having the first form of the S-adensoylmethionine decarboxylase and the fungal organism having the second form of the S-adenosylmethionine decarboxylase are Magnaporthe and the first and the second form of the S-adenosylmethionine decarboxylase are fungal S-adenosylmethionine decarboxylase s.

26. The method of claim 24, wherein the first form of the S-adenosylmethionine decarboxylase is SEQ ID NO:1 or SEQ ID NO:2.

27. The method of claim 24, wherein the fungal organism having the first form of the S-adensoylmethionine decarboxylase and the fungal organism having the second form of the S-adenosylmethionine decarboxylase are Magnaporthe and the first form of the S-adensoylmethionine decarboxylase is SEQ ID NO:1 or SEQ ID NO:2.

28. The method of claim 24, wherein the fungal organism having the first form of the S-adensoylmethionine decarboxylase and the fungal organism having the second form of the S-adenosylmethionine decarboxylase are Magnaporthe, the first form of the S-adensoylmethionine decarboxylase is SEQ ID NO:1 or SEQ ID NO:2, and the second form of the S-adenosylmethionine decarboxylase is a heterologous S-adensoylmethionine decarboxylase.

29. The method of claim 24, wherein the fungal organism having the first form of the S-adenosylmethionine decarboxylase and the fungal organism having the second form of the S-adenosylmethionine decarboxylase are Magnaporthe, the first form of the S-adensoylmethionine decarboxylase is SEQ ID NO:1 or SEQ ID NO:2, and the second form of the S-adenosylmethionine decarboxylase is SEQ ID NO:1 or SEQ ID NO:2 comprising a transposon insertion that reduces or abolishes S-adenosylmethionine decarboxylase activity.

30. A method for identifying a test compound as a candidate for an antibiotic, comprising:
a) providing a fungal organism having a first form of a gene in the polyamine biosynthetic pathway;
b) providing a fungal organism having a second form of said gene in the polyamine biosynthetic pathway, wherein one of the first or the second form of the gene has at least 10% of the activity of a corresponding Magnaportha grisea gene; and
c) determining the growth of the organism having the first form of the gene and the organism having the second form of the gene in the presence of a test compound,
wherein a difference in growth between the two organisms in the presence of the test compound indicates that the test compound is a candidate for an antibiotic.

31. The method of claim 30, wherein the fungal organism having the first form of the gene and the fungal organism having the second form of the gene are Magnaporthe.

32. A method for identifying a test compound as a candidate for an antibiotic, comprising:
a) providing a fungal organism having a first form of a gene in the polyamine biosynthetic pathway;
b) providing a fungal organism having a second form of said gene in the polyamine biosynthetic pathway, wherein one of the first or the second form of the gene has at least 10% of the activity of a corresponding Magnaportha grisea gene; and
c) determining the pathogenicity of the organism having the first form of the gene and the organism having the second form of the gene in the presence of a test compound,
wherein a difference in pathogenicity between the organism and the comparison organism in the presence of the test compound indicates that the test compound is a candidate for an antibiotic.

33. The method of claim 32, wherein the fungal organism having the first form of the gene and the fungal organism having the second form of the gene are Magnaporthe.

34. A method for identifying a test compound as a candidate for an antibiotic, comprising:
a) providing paired growth media containing a test compound, wherein the paired growth media comprise a first medium and a second medium and the second medium contains a higher level of polyamine than the first medium;
b) innoculating the first and the second medium with an organism; and
c) determining the growth of the organism, wherein a difference in growth of the organism between the first and second medium indicates that the test compound is a candidate for an antibiotic.

35. The method of claim 34, wherein the organism is a fungus.

36. The method of claim 34, wherein the organism is Magnaporthe.

37. An isolated nucleic acid comprising a nucleotide sequence that encodes a polypeptide of SEQ ID NO:3.

38. An isolated nucleic acid comprising a nucleotide sequence encoding a polypeptide having at least 50% sequence identity to SEQ ID NO:3 and having at least 10% of the activity of SEQ ID NO:3.

39. An isolated nucleic acid comprising a nucleotide sequence that encodes a polypeptide consisting essentially of the amino acid sequence of SEQ ID NO:3.

40. An isolated polypeptide consisting essentially of the amino acid sequence of SEQ ID NO:3.

41. An isolated polypeptide comprising the amino acid sequence of SEQ ID NO:3.


SEQUENCE LISTING (3 sequences)

SEQ ID NO: 1 - DNA, 1437 bp, Magnaporthe grisea
atgggttgcgagactggaaccaacgagggccagggctacagcttgcccagcgctcctcagctcaccatcaaccacgatgttgcgcaagacctagactctagcggtgcatttgagggacccgagaaactcctcgaggtgtggttcgccccctctcccagtgccctcccgttgggcaccaaggaaaatggcctcaagtcggttccttcagacaactgggttgagatgcttgacattgtcaactgcaagatcctctcggtcgtgcagtcgtctgttgttgatgcatacctcctgtccgagtcgtccatgtttgtcttcccgcacaagatcattctcaagacttgcggaaccacgaccctgcttctgggactcgcccagctcctgcgcatcgccgccgtcgatgccggtttcccggtccacaatgcttcctctgttgaggatgaaaaggccgctgccactccgtaccgcgtcttttacagtcgcaagaacttcctcttcccagaccgccagcgcggcccccaccgcagctggaagcaggaggtcaagtacctcgacagcatgttcgagggtggtagcgcgtacatggtcggcaagatgaacggtgatcattggtacctctacatgacgagccctggctctactgccctcacgcccccgcagacgccgccggctggggaactcatgcgcattcccaccggtcttcagaccgcggcgagtcgtgaggacgacgagacgcttgaagttctgatgacggacttggaccctgagaatgccaagcagttctacctggagcaggccagcgccctggcttgcaagcaggcgacgcttgcccagcaggcaagggaggaggcccatgccgcactggacaaggccgcttccaccgacgagcaattggtctcagaggccctcaccactgaaggccatgccctcggtactgtcgtgtcagacacttgcggcttgtcggacgtgtaccccaagtcaaagtaccctgatgcacgcatcgacgcctacatgtttgagccgtgcggcttttcggcgaacggtgtggttccagctcctcctgacgcgactggtgctcagggcggcaacgagcactactttacggtgcatgtgacaccggagcctaactgttcgtatgcgtcgtttgagactaatgtgcccggtggtcaaaacggccgggagactgccgacatcattggacatgtcgttggcatcttcaagcctggaaggttcagcgtgacccttttcgaaggcaagggccgtcgtggcgagaacggtaccaaggcagaccagaggctgagggttgacaacgttccgggataccgacagctggacaagattgtgcacgagtttgacgactatgatcttgtctttcgcttctaccagcgtgagggctgggtgggcaaggagggtgccagggttggagaggatgatttgtga

SEQ ID NO: 2 - DNA, 1583 bp, Magnaporthe grisea
cataaacgtcttgacaagacaaacatgggttgcgagactggaaccaacgagggccagggctacagcttgcccagcgctcctcagctcaccatcaaccacgatgttgcgcaagacctagactctagcggtgcatttgagggacccgagaaactcctcgaggtgtggttcgccccctctcccagtgccctcccgttgggcaccaaggaaaatggcctcaagtcggttccttcagacaactgggttgagatgcttgacattgtcaactgcaagatcctctcggtcgtgcagtcgtctgttgttgatgcatacctcctgtccgagtcgtccatgtttgtcttcccgcacaagatcattctcaagacttgcggaaccacgaccctgcttctgggactcgcccagctcctgcgcatcgccgccgtcgatgccggtttcccggtccacaatgcttcctctgttgaggatgaaaaggccgctgccactccgtaccgcgtcttttacagtcgcaagaacttcctcttcccagaccgccagcgcggcccccaccgcagctggaagcaggaggtcaagtacctcgacagcatgttcgagggtggtagcgcgtacatggtcggcaagatgaacggtgatcattggtacctctacatgacgagccctggctctactgccctcacgcccccgcagacgccgccggctggggaactcatgcgcattcccaccggtcttcagaccgcggcgagtcgtgaggacgacgagacgcttgaagttctgatgacggacttggaccctgagaatgccaagcagttctacctggagcaggccagcgccctggcttgcaagcaggcgacgcttgcccagcaggcaagggaggaggcccatgccgcactggacaaggccgcttccaccgacgagcaattggtctcagaggccctcaccactgaaggccatgccctcggtactgtcgtgtcagacacttgcggcttgtcggacgtgtaccccaagtcaaagtaccctgatgcacgcatcgacgcctacatgtttgagccgtgcggcttttcggcgaacggtgtggttccagctcctcctgacgcgactggtgctcagggcggcaacgagcactactttacggtgcatgtgacaccggagcctaactgttcgtatgcgtcgtttgagactaatgtgcccggtggtcaaaacggccgggagactgccgacatcattggacatgtcgttggcatcttcaagcctggaaggttcagcgtgacccttttcgaaggcaagggccgtcgtggcgagaacggtaccaaggcagaccagaggctgagggttgacaacgttccgggataccgacagctggacaagattgtgcacgagtttgacgactatgatcttgtctttcgcttctaccagcgtgagggctgggtgggcaaggagggtgccagggttggagaggatgatttgtgatggttgtgaattggcgcttctggtttggattctttacgtgttatatcaagtattgaatattttcaataatccgttcctctcatcattgcatgtagcatttagttctttcattgcagcacatt

SEQ ID NO: 3 - protein, 478 aa, Magnaporthe grisea
MGCETGTNEGQGYSLPSAPQLTINHDVAQDLDSSGAFEGPEKLLEVWFAPSPSALPLGTKENGLKSVPSDNWVEMLDIVNCKILSVVQSSVVDAYLLSESSMFVFPHKIILKTCGTTTLLLGLAQLLRIAAVDAGFPVHNASSVEDEKAAATPYRVFYSRKNFLFPDRQRGPHRSWKQEVKYLDSMFEGGSAYMVGKMNGDHWYLYMTSPGSTALTPPQTPPAGELMRIPTGLQTAASREDDETLEVLMTDLDPENAKQFYLEQASALACKQATLAQQAREEAHAALDKAASTDEQLVSEALTTEGHALGTVVSDTCGLSDVYPKSKYPDARIDAYMFEPCGFSANGVVPAPPDATGAQGGNEHYFTVHVTPEPNCSYASFETNVPGGQNGRETADIIGHVVGIFKPGRFSVTLFEGKGRRGENGTKADQRLRVDNVPGYRQLDKIVHEFDDYDLVFRFYQREGWVGKEGARVGEDDL